(19) United States
(12) Patent Application Publication
(10) Pub. No.: US 2024/0225874 A1
(43) Pub. Date: Jul. 11, 2024

(54) VERTICALLY ORIENTED BAND FOR STOMACH

(71) Applicant: Advanced Bariatric Technology, LLC, Coral Gables, FL (US)

(72) Inventors: Moises Jacobs, Miami, FL (US); Moises Jacobs, III, Miami, FL (US)

(73) Assignee: Advanced Bariatric Technology, LLC, Coral Gables, FL (US)

(21) Appl. No.: 18/615,902

(22) Filed: Mar. 25, 2024

Related U.S. Application Data
(63) Continuation of application No. 16/531,974, filed on Aug. 5, 2019, now Pat. No. 11,938,049.
Application No. 16/531,974 is a continuation of application No. 15/605,812, filed on May 25, 2017, now Pat. No. 10,369,036.
Application No. 15/605,812 is a continuation of application No. 14/531,300, filed on Nov. 3, 2014, now Pat. No. 9,814,614.
Application No. 14/531,300 is a continuation of application No. 11/984,452, filed on Nov. 19, 2007, now Pat. No. 8,920,305.
Application No. 11/984,452 is a continuation-in-part of application No. 11/797,537, filed on May 4, 2007, now abandoned.
(60) Provisional application No. 60/881,138, filed on Jan. 19, 2007.

(51) Int. Cl. A61F 5/00; A61B 17/122

(52) U.S. Cl. CPC A61F 5/0086; A61B 17/122

(57) ABSTRACT

An adjustable band or clamp or non-adjustable clamp is placed about the greater curvature of the stomach in a vertical orientation. The band or clamp compartmentalizes the stomach between a small vertical pouch and the fundus and body of the stomach. The fundus and body of the stomach are excluded from nutrients and are separated from a long narrow channel where the food travels. A small passage at the level of the antrum allows gastric juices to empty from the fundus and body of the stomach. The clamp may be applied during open surgery in laproscopic surgery or using a single port technique, or through any natural orifice in NOTES (Natural Orifice Transluminal Endoscopic surgery) or using a hybrid surgical technique.

### CROSS-REFERENCE TO RELATED APPLICATIONS

Pursuant to 35 U.S.C. § 120, this application is a continuation of U.S. patent application Ser. No. 16/531,974, entitled “Vertically Oriented Band for Stomach,” filed Aug. 5, 2019, and naming Moises Jacobs and Moises Jacobs III as inventors, which is a continuation of U.S. patent application Ser. No. 15/605,812, entitled “Vertically Oriented Band for Stomach,” filed May 25, 2017, and naming Moises Jacobs and Moises Jacobs III as inventors, which is a continuation of, U.S. patent application Ser. No. 14/531,300, entitled “Vertically Oriented Band for Stomach,” filed Nov. 3, 2014, and naming Moises Jacobs and Moises Jacobs III as inventors, which is a continuation of, U.S. patent application Ser. No. 11/984,452, entitled “Vertically Oriented Band for Stomach,” filed Nov. 19, 2007, and naming Moises Jacobs and Moises Jacobs III as inventors, which is a continuation-in-part of U.S. patent application Ser. No. 11/797,537, entitled “Vertically Oriented Band for Stomach,” filed May 4, 2007, and naming Moises Jacobs and Moises Jacobs III as inventors, which claims the benefit of US Provisional Application Number 60/881,138, entitled “Vertically Oriented Band for Stomach,” filed Jan. 19, 2007, and naming Moises Jacobs as inventor, all of which are incorporated by reference for all purposes.

### BACKGROUND

For patients whose obesity presents an immediate serious health risk, surgical procedures are available to promote weight loss. Two of the most common surgical procedures are gastric bypass and gastric band. During gastric bypass, the stomach is made smaller and food bypasses part of the small intestine. The smaller size stomach causes the patient to eat less and the bypass of the small intestines leads to less calories being absorbed by the body.

In the most common type of gastric bypass surgery, roux-en-y, a small pouch is formed at the top of the stomach using staples. The smaller stomach is connected to the middle portion of the small intestines bypassing the upper portion of the small intestines.

Devices have been developed to form the smaller stomach from the patient's original stomach. One such device is disclosed in U.S. patent Publication No. 2002/0022851 to Kalloo et al. (Kalloo). Kalloo discloses a loop 80 reducing the volume of the gastric cavity. A feeder line is pulled to reduce the diameter of the loop and collapse the walls of the stomach to define a smaller pouch.

U.S. patent Publication No. 2006/0157067 to Saadat et al. (Saadat) discloses the use of tissue anchors to form a gastric pouch acting as a restriction to the passage of fluids and food. U.S. Pat. No. 5,345,949 to Shlain (Shlain) discloses a clip placed across the fundus of the stomach to restrict the inlet chamber or proximal pouch. Likewise, U.S. Pat. No. 6,869,438 to Chao (Chao) discloses a gastric partitioning clip creating a stomach pouch from the stomach to restrict the amount of food intake.

It is an object of the invention to provide a device for separating the stomach into two compartments but allowing communication between the compartments.

It is another object of the invention to provide a device for forming a smaller stomach pouch, the size of the pouch being tailored to the patient's individual circumstances.

It is another object of the invention to provide a procedure creating a small stomach pouch to limit intake of food separate from the stomach but allowing gastric juices from the excluded stomach to flow into the pouch.

It is still another object of the invention to provide a system for creating a small pouch from the main stomach that is reversible.

It is still another object of the invention to alter the production of hormones, enzymes and chemicals that affect metabolism, energy levels, hunger, digestion, absorption of nutrients, weight loss, maintenance or gain that may be affected by exclusion of the gastric fundus and body of the stomach.

These and other objects of the invention will become apparent after reading the disclosure of the invention.

### SUMMARY

An adjustable band or clamp or non-adjustable clamp is placed about the greater curvature of the stomach in a vertical orientation. The band or clamp completely compartmentalizes the stomach between a small vertical pouch and the fundus and body of the stomach. The fundus and body of the stomach are excluded from nutrients and are separated from a long narrow channel where the food travels. A small passage at the level of the antrum allows gastric juices to empty from the fundus and body of the stomach. The clamp may be applied during open surgery in laproscopic surgery or using a single port technique, or through any natural orifice in NOTES (Natural Orifice Transluminal Endoscopic surgery) or using a hybrid surgical technique.

### BRIEF DESCRIPTION OF DRAWINGS

FIG. 1 is a view of the band applied to a stomach;

FIG. 2 is a cross-sectional view of an inflatable clamp useable with the invention;

FIG. 3 is a view of a non-adjustable clamp used with the invention;

FIG. 4 is a detailed view of the strap attachment to the band;

FIG. 5 is a perspective view of a second embodiment of the band; and

FIG. 6 is a view of the band in FIG. 5 applied to a stomach.

### DETAILED DESCRIPTION OF THE DRAWINGS

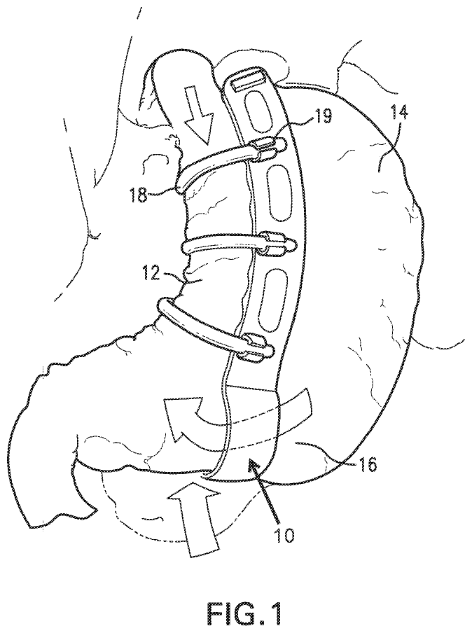

In FIG. 1, a stomach having the band 10 applied can be seen dividing the stomach into the pouch 12 and fundic and body area 14. Food traveling down the esophagus enters the pouch 12 and exits into the antrum. The band 10 applies pressure against the sides of the stomach to separate the stomach into the two compartments, but does not apply pressure to the stomach walls at the bottom part of the stomach. This creates a passage 16 allowing flow of gastric juices from the fundic and body area 14 into the antrum. Food will not enter the fundic and body of the stomach through this passage, however. At least one horizontal strap 18 maybe used to secure the band in place. The straps may be adjustable and may apply pressure sufficient to impact the size and function of the pouch 12.

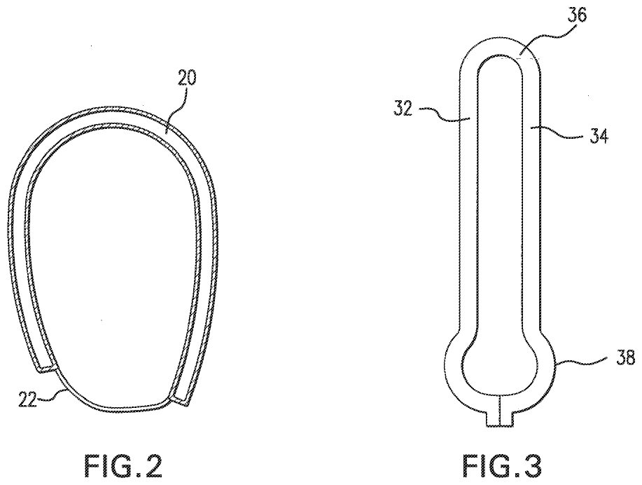

FIG. 2 shows an embodiment of the clamp having an inflatable chamber 20 and a connecting section 22. The clamp is placed about the stomach in a vertical orientation to separate the stomach into the two compartments and inflated. The clamp may have an asymmetrically placed chamber 20, that when inflated applies pressure on the stomach to seal the two compartments from one another except for the passage 16. The connecting section 22, being not inflated, does not apply pressure to the bottom portion of the stomach, allowing for the formation of the passage 16. In addition, the clamp may have an asymmetrically placed inflatable chamber that faces the lesser curvature side of the stomach, that when inflated or deflated only alters the lumen of the vertical compartment through which the nutrients pass and does not play a role in the creation of the two compartments.

FIG. 3 shows the rigid clamp embodiment having a U portion formed by two legs 32, 34 connected by a bight portion 36. When the clamp is placed on the stomach, the bight portion 36 fits over the top of the stomach with the legs 32, 34 applying enough pressure to collapse the walls of the stomach against one another to create the two compartments. The legs 32, 34 mayor may not extend the full vertical extent of the stomach to allow for the creation of the passage 16. The legs are attached by a connector 38. When applied to the stomach, the legs serve to push the sides of the stomach together to form a complete seal but the connector allows for the formation of a passage between the two compartments. The clamp may be adjustable. The legs of the clamp may be made or adjusted to any length depending on the size of the stomach the legs can be made shorter or longer. The two legs may be connected by a magnetic coupler rather than a solid bight portion but may not be connected at all at the proximal end of the stomach. The apposition of the clamp legs about the stomach must be accomplished without sufficient force to cause ischemia of the gastric walls when the legs are closed.

Besides a clip, the vertical band may be formed as or with an inflatable balloon, as discussed with reference to FIG. 2. The orientation of the balloon is such that, upon inflation, the balloon bulges to the left to decrease the size of the compartment 12. The bottom portion may or may not be inflatable. The balloon may be attached to a tube exiting the body so that the balloon may be adjusted without the need for invasive surgery.

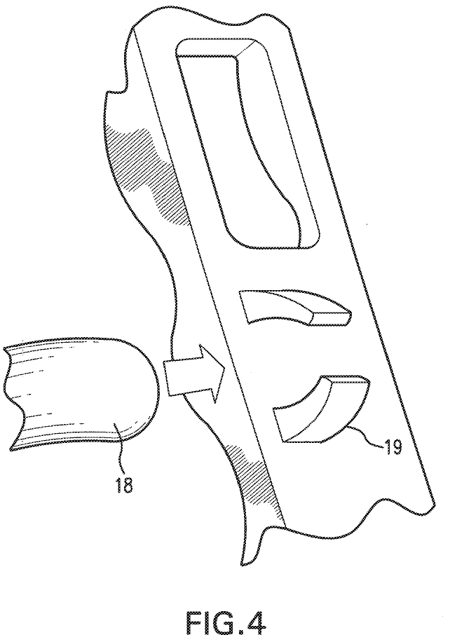

Straps 18 can be secured to the band in any number of conventional ways. One possible way in which to secure the straps to the band is depicted in FIG. 4. The band engages and is secured by clips 19 which extend outwardly from the band. This arrangement allows the straps to be tightened by being pulled through the clip and, if desired, the straps can be released for the removal of the band. These straps may also have an inflatable chamber and may be adjustable so as to also increase or decrease the lumen of the vertical compartment through which the nutrients pass.

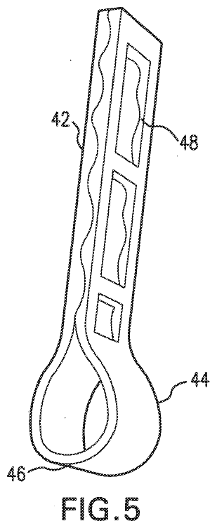

An alternative construction of the band is seen in FIG. 5. In this embodiment, the band has a first section 42 having two parallel arms and a second section with two space members so that, when applied to a stomach, the passageway 16 is formed. The clamp itself may be curved to allow for better accommodation about the lesser curvature. The arms may be straight, curved or undulating. The surface may be smooth or serrated. The arms of the first section 42 are resiliently biased against one another and are spaced from one another in order that, when applied, the first section maintains the walls of the stomach together to separate the stomach into the first and second compartments. The pressure applied must be enough that the two compartments are formed but not so much that the walls of the stomach are damaged or compromise the blood supply. The section 44 is connected together by a section 46 acting as a hinge. This allows the arms of the first section 42 to be separated from one another in order that the band may be applied. Conversely, it is possible to have the two arms of the first section 42 hinged to one another and the two arcuate portions forming the second section 44 not connected to one another.

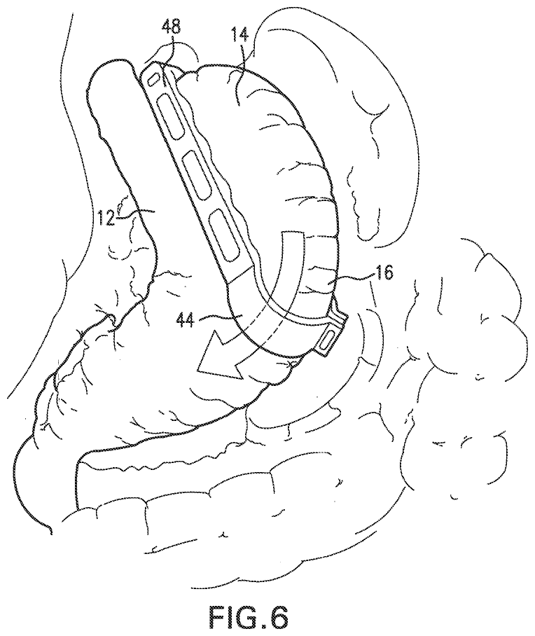

The band of FIG. 5 applied to stomach is seen in FIG. 6. Seen here as the first section 42 extending along the stomach to separate the stomach into two compartments, including pouch 12 and fundic and body area 14, whereas the second section has arcuate arms forming a passage 16. At least one of the arms of the first section is provided with apertures 48. The apertures, which may be large or small, allow part of the stomach wall to enter the aperture to help prevent movement of the band once it has been applied.

There are many ways in which the clamp can be applied including Natural orifice transluminal endoscopic surgery (NOTES) and the combination of NOTES and an assistant trochar placed in to the abdominal cavity. Combinations include any combination of the conventional, laproscopic, NOTES and one port techniques. The NOTES technique includes transgastric, transvaginal, transrectal, transcolonic and combinations of these. Another possibility is the one port technique wherein one port is used for the introduction of several instruments. The one port technique encompasses a one port abdominal (including umbilical), perineal, retroperitoneal approaches and combinations of these.

To facilitate application of the band, a bougie may be utilized in any suitable manner, such as placed transorally, transgastrically or transintestinally. The bougie, having a vacuum suction apparatus, collapses the stomach wall to align and help the placement the clamp. To help with alignment and placement of the clamp, the bougie may have magnets to mate with the magnets or metallic areas when the clamp is provided with such. Also, the band may be made of bioabsorbable material to negate the need to remove it.

While the invention has been described with reference to preferred embodiments, various modifications would be apparent to one of ordinary skill in the art. The invention encompasses such variations and modifications.

What is claimed is:

1. A method of installing a stomach band onto a stomach of a body, the method comprising:
providing the stomach band that may be positioned in an open position or in a closed position;
inserting the stomach band into the body through an opening;
positioning a first arm of the stomach band on a first side of the stomach, and positioning a second arm of the stomach band on a second side of the stomach while the stomach band is in the open position; and
closing and securing the stomach band into the closed position around the stomach to form a first compartment and a second compartment within the stomach with a partitioning section of the stomach band, and to form a fluid passage within the stomach with a passage-forming section of the stomach band such that, when the stomach band is secured on the stomach in the closed position, the first compartment of the stomach is in fluid communication with the second compartment of the stomach through the fluid passage of the stomach;
wherein the partitioning section of the stomach band generally obstructs flow of a fluid from the second compartment of the stomach to the first compartment of the stomach when the stomach band is installed in the closed position around the stomach,
wherein the fluid contained within the second compartment of the stomach may flow through the fluid passage into the first compartment of the stomach while the stomach band is installed in the closed position around the stomach, and
wherein the stomach band includes:
the partitioning section comprising the first arm and the second arm; and
the passage-forming section comprising a first curved member positioned adjacent an end of the first arm, and a second curved member positioned adjacent an end of the second arm,
wherein, when the stomach band is secured to the stomach in the closed position, the first arm of the partitioning section is separated from the second arm of the partitioning section by a first distance,
wherein, when the stomach band is secured to the stomach in the closed position, an apex of a curve of the first curved member and an apex of a curve of the second curved member are separated by a second distance, and
wherein the second distance is greater than the first distance.

2. The method of installing a stomach band of claim 1, wherein closing and securing the stomach band into the closed position around the stomach includes coupling a second end of the first arm and a second end of the second arm.

3. The method of installing a stomach band of claim 1, wherein the fluid passage is formed at or adjacent an antrum portion of the stomach.

4. The method of installing a stomach band of claim 1, wherein the stomach band includes a padding material on at least a portion of the first arm.

5. The method of installing a stomach band of claim 4, wherein the padding material is a silicone sleeve.

6. The method of installing a stomach band of claim 1, wherein at least a portion of the first arm and at least a portion of the second arm are curved to conform the first arm and the second arm to a lesser curvature of the stomach.

7. The method of installing a stomach band of claim 1, wherein the inserting the stomach band into the body through an opening includes natural orifice transluminal endoscopic surgery (NOTES).

8. The method of installing a stomach band of claim 1, wherein the inserting the stomach band into the body through an opening includes one of transgastric surgery, transvaginal surgery, transrectal surgery, and transcolonic surgery.

9. The method of installing a stomach band of claim 1, wherein the inserting the stomach band into the body through an opening includes placing an assistant trochar into an abdominal cavity of the body.

10. The method of installing a stomach band of claim 1, wherein the first curved member is coupled to the second curved member.

11. A method of partially partitioning a stomach of a body, the method comprising:
providing a stomach band that may be provided in an open position and in a closed position;
inserting the stomach band into the body through an opening;
positioning a partitioning section of the stomach band on the stomach; and
closing and securing the stomach band in the closed position around the stomach to partition a portion of the stomach with the partitioning section of the stomach band, wherein, when the stomach band is secured on the stomach in the closed position, the stomach is not fully partitioned;
wherein the stomach band when in the closed position includes:
a first arm having a first end and a second end;
a second arm having a first end and a second end; and
a bight having a hinge positionable between the open position and the closed position, the bight positioned adjacent the second end of the first arm and the second end of the second arm,
wherein at least a portion of the first arm and at least a portion of the second arm comprise the partitioning section of the stomach band when the stomach band is secured in the closed position around the stomach,
wherein the partitioning section generally obstructs flow of fluid contained within the partitioned portion of the stomach when the stomach band is installed in the closed position around the stomach, and
wherein at least a portion of the bight comprises a passage-forming section, and the passage-forming section defines a fluid passage through which a fluid contained within the stomach may flow when the stomach band is installed in the closed position around the stomach.

12. The method of claim 11, wherein the inserting the stomach band into the body through an opening includes natural orifice transluminal endoscopic surgery (NOTES).

13. The method of claim 11, wherein the inserting the stomach band into the body through an opening includes placing an assistant trochar into an abdominal cavity of the body.

14. The method of claim 11, wherein the stomach band is positioned on the stomach in a substantially vertical position.

15. The method of claim 11, wherein the stomach band includes a padding material on at least a portion of the first arm.

16. The method of claim 15, wherein the padding material is a silicone sleeve.

17. The method of claim 11, wherein the hinge of the bight is biased such that the first arm is resiliently biased toward the second arm.

18. The method of claim 11, wherein at least a portion of the first arm and at least a portion of the second arm are curved to conform the partitioning section of the stomach band to a lesser curvature of the stomach.

19. The method of claim 11, wherein the stomach band further comprises a coupling configured to couple the first end of the first arm and the first end of the second arm.

20. The method of claim 11, wherein the stomach is not fully occluded at an antrum portion of the stomach when the stomach band is secured in the closed position around the stomach.
